(19) United States
(12) Patent Application Publication
(10) Pub. No.: US 2011/0022064 A1
(43) Pub. Date: Jan. 27, 2011

(54) ACCESS APPARATUS INCLUDING HINGED SUTURE TRAPS

(75) Inventor: Andrew Barnes, Naugatuck, CT (US)

Correspondence Address: Tyco Healthcare Group LP;d/b/a Covidien, 555 Long Wharf Drive, Mail Stop 8-N1, Legal Department, New Haven, CT, 06511, US

(73) Assignee: TYCO Healthcare Group LP

(21) Appl. No.: 12/774,148

(22) Filed: May 5, 2010

Related U.S. Application Data
(60) Provisional application No. 61/228,638, filed on Jul. 27, 2009.

(51) Int. Cl. A61B 17/04

(52) U.S. Cl. 606/148

(57) ABSTRACT

An access apparatus includes an access member defining a longitudinal axis and having a longitudinal passage adapted to permit passage of a surgical suture utilized in performing a surgical procedure, a housing cap mountable with respect to the access member, and defining a cap opening in communication with the longitudinal passage of the access member to permit passage of the surgical suture and a suture trap mounted to the housing cap. The suture trap is pivotally movable, e.g., via a hinge, relative to the housing cap between a first position where a suture is positionable with respect to the housing cap, and a second position where the suture trap is secured relative to the housing cap and retains a suture segment of the suture in a predetermined relation. The housing cap may include a suture groove in communication with the cap opening and in general alignment with the suture trap. The suture groove is arranged to accommodate the surgical suture extending through the cap opening.

### CROSS REFERENCE TO RELATED APPLICATION

The present application claims the benefit of and priority to U.S. Provisional Application Ser. No. 61/228,638 filed on Jul. 28, 2009, the entire contents of which are incorporated herein by reference.

### BACKGROUND

1. Technical Field

The present disclosure relates to an access apparatus for providing access into a patient's body. In particular, the present disclosure relates to an access apparatus with suture managing capabilities to assist the clinician in the performance of surgical procedures involving application of sutures to repair hard and/or soft tissue.

2. Description of Related Art

Trocars and other surgical portal apparatus are known, as are myriad procedures that may be preformed using such assemblies. Many of the minimally invasive procedures performed through portal apparatus necessitate or are simplified by the use of one or more sutures passing through the surgical portal apparatus. Sutures extending into a body cavity through a surgical portal apparatus may be used to, for example, temporarily retain tissue, manipulate tissue, anchor tissue or operate peripheral devices. In an attempt to reduce the number of incision sites required to complete a given surgical procedure, a single surgical portal apparatus may be used to pass one or more sutures into a body cavity, in addition to providing access for one or more devices. A single anchor device may have numerous suture extending from the device and through the surgical portal apparatus. The sutures extending through the surgical portal apparatus may become tangled as each is manipulated or as one or more instruments are inserted and withdrawn from the assembly. Also, a surgeon may confuse the suture ends during the course of a surgery. Tangling or confusion of the suture ends may unnecessarily complicate the procedure and increase time necessary to complete the procedure.

Therefore, it would be beneficial to have a surgical portal apparatus that includes a system for managing sutures during a surgical procedure such as a laparoscopic, arthroscopic, endoscopic and/or orthopedic procedure.

### SUMMARY

In accordance with one embodiment, an access apparatus includes an access member defining a longitudinal axis and having a longitudinal passage adapted to permit passage of a surgical suture utilized in performing a surgical procedure, a housing cap mountable with respect to the access member, and defining a cap opening in communication with the longitudinal passage of the access member to permit passage of the surgical suture and a suture trap mounted to the housing cap. The suture trap is pivotally movable, e.g., via a hinge, relative to the housing cap between a first position where a suture is positionable with respect to the housing cap, and a second position where the suture trap is secured relative to the housing cap and retains a suture segment of the suture in a predetermined relation. The housing cap may include a suture groove in communication with the cap opening and in general alignment with the suture trap. The suture groove is arranged to accommodate the surgical suture extending through the cap opening.

The suture trap may be pivotally mounted to the housing cap. Means for releasably locking the suture trap in the second position may be provided. The suture trap may include a stopper and the housing cap may include a recess component. The stopper may be adapted for reception within the recess component to releasably secure the suture trap in the second position. The stopper may be dimensioned to establish a friction fit within the recess component of the housing cap. A plurality of suture traps may be arranged in spaced relation with respect to the housing cap.

Each suture trap may be adapted to secure a suture segment of a surgical suture in the predetermined relation. The housing may include a plurality of suture grooves in communication with the cap opening and in general alignment with respective suture traps. The suture grooves may be arranged to accommodate the surgical suture extending through the cap opening.

One of the housing cap and the suture trap may include a suture retaining channel for receiving a segment of the suture extending from the cap opening. The suture is secured within the suture retaining channel in fixed relation relative to the housing cap.

An access housing may be mounted to the access member with the housing cap being mountable with respect to the access housing. The housing cap may be releasably mountable with respect to the access housing.

An instrument seal disposed may be in mechanical cooperation with the housing. The instrument seal is adapted to establish a substantial seal about an object introduced through the access member.

### BRIEF DESCRIPTION OF THE DRAWINGS

Various embodiments of the present disclosure are described herein with reference to the drawings wherein:

FIG. 1 is a side cross-sectional view of an access apparatus including a housing with suture traps shown in an open position;

FIG. 2 is a perspective view of a housing cap of the housing of the access apparatus of FIG. 1;

FIG. 3 is an enlarged cross-sectional view of one embodiment of the suture trap illustrating the suture trap in a closed position;

FIG. 4 is an enlarged cross-sectional view of one embodiment of the suture trap illustrating the suture trap in a closed position;

FIG. 5 is an enlarged cross-sectional view of another embodiment of the suture trap illustrating the suture trap in a closed position; and

FIG. 6 is an enlarged cross-sectional view of another embodiment of the suture trap illustrating the suture trap in a closed position.

### DETAILED DESCRIPTION

The access apparatus of the present disclosure provides a substantially fluid-tight seal between a body cavity of a patient and the outside atmosphere. The access apparatus of the present disclosure is configured to receive surgical instruments of varying diameters and is also configured to retain sutures therein to help assist in suture identification and prevent or minimize suture entanglement. Included among the various procedures contemplated by the present disclosure are endoscopic, laparoscopic, arthroscopic, orthopedic, etc.

The access apparatus of the present disclosure contemplates the introduction of various types of instrumentation during the particular procedure. Examples of instrumentation include, but are not limited to, clip appliers, graspers, dissectors, retractors, staplers, laser probes, photographic devices, endoscopes and laparoscopes, tubes, anchors, anchor drives, etc. Such instruments will collectively be referred to as “instruments” or “instrumentation” or “surgical objects.”

In addition to instrumentation, the access apparatus permits the passage of one or more sutures. When one or more sutures are used in conjunction with an arthroscopic procedure, the following criteria is significant to the overall success of the surgery 1) suture identification of the sutures extending through the access apparatus; and 2) minimization of the potential of suture entanglement.

In the following description, as is traditional, the term “proximal” refers to the portion of the device closer to the operator while the term “distal” refers to the portion of the device farther from the operator.

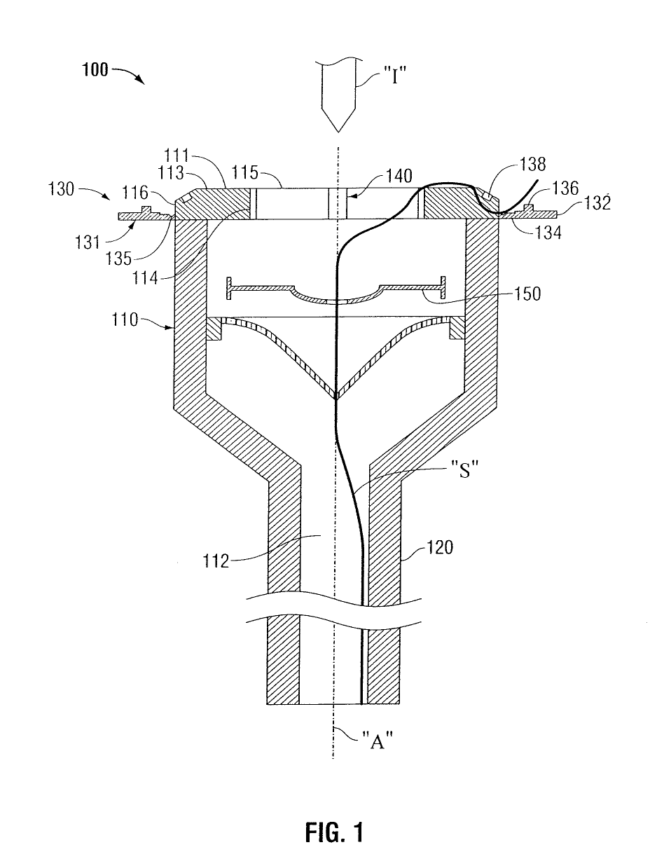

Referring now to the drawings, FIG. 1 illustrates an access apparatus 100 that defines a longitudinal axis “A” and includes a housing 110 and an access member 120. Housing 110 has a housing cap 111 adapted to detachably engage a proximal end of housing 110, e.g., through a bayonet lock, threaded connection, or like mechanical means. Housing cap 111 may include one suture trap 130 or may include a plurality of suture traps 130a, 130b, 130c, 130d, etc. Housing cap 111 has a cap opening 115 and access member defines a longitudinal passageway 112. Cap opening 115 and longitudinal passageway 112 are generally aligned with respect to the longitudinal axis “A” to permit passage of surgical objects such as instruments “I” and sutures “S” utilized in connection with the procedure.

Housing cap 111 of housing 110 includes grooves 140 defined within an inner wall 114 of proximal surface 113 of the housing cap 111. Each grooves 140 is configured to retain at least a portion or segment of a suture “S” therein. The number of suture traps 130 provided may depend on the number of sutures “S” to be used during the surgical procedure. Additionally, the number of grooves 140 defined within inner wall 114 of proximal surface 113 of housing cap 111 may correspond with the number of suture traps 130, such that each suture “S” may rest in a groove 140 and be retained by a suture trap 130. Grooves 140 are generally radially aligned with suture traps 130.

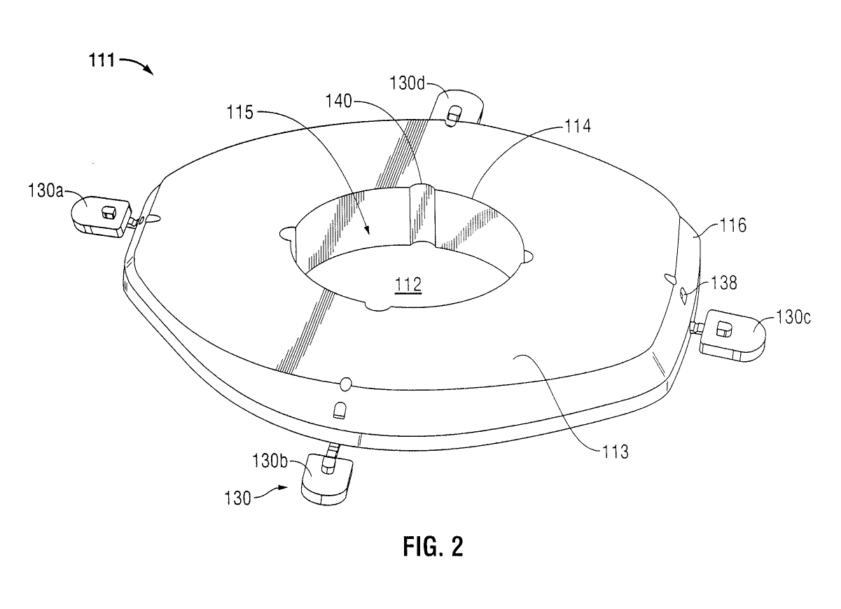

As shown in FIGS. 1 and 2, suture trap 130 includes a head 132 having a stopper 136 and an attachment member 134. Attachment member 134 is disposed in mechanical cooperation with housing cap 111 and may be a hinge segment monolithic with the housing cap 111 or a separate component attached via a pivot pin or the like. Head 132 is adapted to be releasably secured relative to housing cap 111 by reception of stopper 136 within corresponding recess 138 in the outer surface of housing cap 111. Stopper 136 of head 132 may mechanically engage recess component 138 via a friction fit, snap fit or the like, and may be releasably connected to enable the selective locking and release of suture trap 130 relative to housing cap 111. FIGS. 1 and 2 illustrate suture trap 130 is in an open position (i.e., stopper 136 is not engaged with recess component 138). It should be recognized that, while FIGS. 1 and 2 illustrate stopper 136 being disposed on head 132 and recess 138 being disposed on the outer surface of housing cap 111, the present invention contemplates that these components may be reversed, e.g., stopper 136 may instead be disposed on the outer surface of housing cap 111 and recess 138 may instead be disposed on head 132 such that stopper 136 of outer surface of housing cap 111 may mechanically engage recess component 138 of head 132 via a friction fit, snap fit or the like.

With continued reference to FIG. 1, a suture “S” is shown disposed through longitudinal passageway 112. A portion of suture “S” rests within groove 140. Grooves 140 may be of sufficient depth to accommodate the diameter of the suture “S”. FIG. 3 depicts suture trap 130 in a closed position (i.e., stopper 136 is mechanically engaged with recess component 138). In one embodiment, a segment of Suture “S” is at least partially retained within a suture channel 135 formed in attachment member 134. The suture trap 130 is configured to substantially hold the suture “S” in place when the suture trap 130 is in the closed position. In particular, the attachment member 134 of suture trap 130 and outer wall 116 of top portion 111 of housing 110 secure the suture “S” therebetween. When desired, suture trap 130 may be released (i.e., stopper 136 is disengaged from recess component 138) back to the open position (FIGS. 1 and 2), thereby releasing suture “S” from suture channel 135. Suture trap 130 maintains at least a suture segment of the suture “S” in predetermined relation relative to housing 110. This in conjunction with the spacing of suture traps 130 about housing cap 111 maintains the sutures “S” in spaced relation thereby minimizing entanglement while also assisting the clinician in suture identification. For example, the clinician will be able to assign a particular suture to a corresponding suture trap 130 thereby enabling the clinician to retrieve the appropriate suture “S” needed during the sequence of the surgical procedure. Visual indicia may be assigned to each suture trap 130 to further assist the surgeon in identifying the sutures “S”. Suture traps 130 may be identified by, e.g., numbers or labels, e.g., 1,2,3,4 (FIG. 2) on the upper surface of housing cap 111.

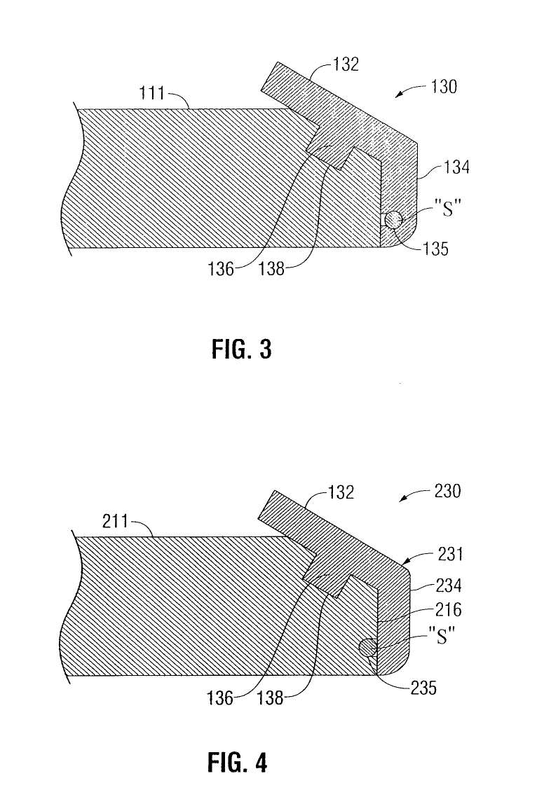

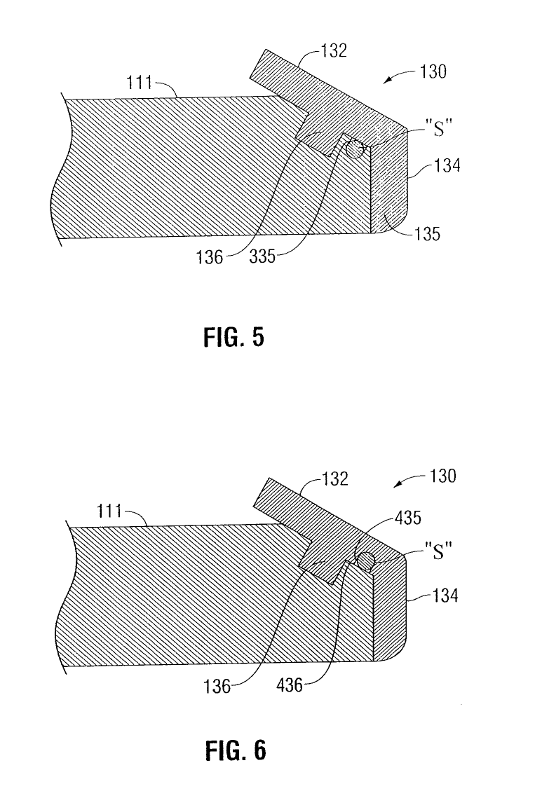

Referring now to FIG. 4, another embodiment of a suture trap 230 is shown in a closed position (i.e., stopper 136 is mechanically engaged with recess component 138). Stopper 136 of head 132 mechanically engages (e.g. via friction fit) recess component 138. A suture channel 235 is disposed within an outer wall 216 of a housing cap 211. Suture “S” may be disposed within suture channel 235 and substantially held in place by suture trap 230. In particular, attachment member 234 may engage the Suture “S” and secure the Suture “S” between the attachment member 234 and the outer wall of the housing cap 211. When it is desired, movable portion 231 may be released (i.e., stopper 136 is disengaged from recess component 138) back to the open position (FIGS. 1 and 2), thereby releasing suture “S” from suture channel 235. FIG. 5 illustrates another embodiment where a suture retaining groove or channel 335 is disposed within an upper surface of housing 110. The Suture “S” is secured or retained within the channel 335 by engagement of head 132 with the suture “S”. Head 132 traps or secures the Suture “S” within channel 235 to fix the suture segment relative to the housing cap 111. As a further alternative, a suture retaining channel 435 may be formed within head 132 as shown in phantom in FIG. 6, and releasably secured a suture “S” against the outer surface 436 of the housing cap.

Referring again to FIG. 1, the access apparatus 100 may include one or more instrument seals 150 disposed within longitudinal passageway 112 and in mechanical cooperation with housing 110. Instrument seal 150 is configured to create a substantially fluid-tight seal around an instrument “I” introduced through the seal 150. Instrument seal 150 may also accommodate one or more sutures “S” passing therethrough. One suitable instrument seal is disclosed in commonly assigned U.S. Pat. No. 6,702,787 to Racenet, the entire contents of which disclosure is incorporated by reference herein.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An access apparatus comprising:
an access member defining a longitudinal axis and having a longitudinal passage, the longitudinal passage being adapted to permit passage of a surgical suture utilized in performing a surgical procedure;
a housing cap mountable with respect to the access member, the housing cap defining a cap opening in communication with the longitudinal passage of the access member to permit passage of the surgical suture; and
a suture trap pivotally mounted to the housing cap, the suture trap being movable relative to the housing cap between a first position where a suture is positionable with respect to the housing, and a second position where the suture trap is secured relative to the housing cap and retains a suture segment of the suture in a predetermined relation.

2. The access apparatus of claim 1 wherein the housing cap includes a suture groove in communication with the cap opening and in general alignment with the suture trap, the suture groove arranged to accommodate the surgical suture extending through the cap opening.

3. The access apparatus of claim 1 wherein the suture trap is pivotally mounted to the housing cap via a hinge.

4. The access apparatus of claim 3 including means for releasably locking the suture trap in the second position.

5. The access apparatus of claim 3 wherein the suture trap includes a stopper and the housing cap includes a recess component, the stopper being adapted for reception within the recess component to releasably secure the suture trap in the second position.

6. The access apparatus of claim 5 wherein the stopper is dimensioned to establish a friction fit within the recess component of the housing cap.

7. The access apparatus of claim 3 including a plurality of suture traps arranged in spaced relation with respect to the housing cap, each suture trap adapted to secure a suture segment of a surgical suture in the predetermined relation.

8. The access apparatus of claim 7 wherein the housing cap includes a plurality of suture grooves in communication with the cap opening and in general alignment with respective suture traps, the suture groove arranged to accommodate the surgical suture extending through the cap opening.

9. The access apparatus of claim 3 wherein one of the housing cap and the suture trap includes a suture retaining channel for receiving a segment of the suture extending from the cap opening, the suture being secured within the suture retaining channel in fixed relation relative to the housing cap.

10. The access apparatus of claim 3 including an access housing mounted to the access member, the housing cap being mountable with respect to the access housing.

11. The access apparatus of claim 10 wherein the housing cap is releasably mountable with respect to the access housing.

12. The access apparatus of claim 1 further comprising an instrument seal disposed in mechanical cooperation with the housing.
